(19) United States
(12) Patent Application Publication
(10) Pub. No.: US 2022/0218315 A1
(43) Pub. Date: Jul. 14, 2022

(54) ROBOT AND SYSTEM FOR A MEDICAL OPERATION OF CORONA VIRUSES SAMPLING

(71) Applicant: Shenzhen Institute of Gratrics, Shenzhen (CN)

(72) Inventors: Quanquan Liu, Shenzhen (CN); Zhengzhi Wu, Shenzhen (CN); Chunbao Wang, Shenzhen (CN); Xin Zhang, Shenzhen (CN); Lihong Duan, Shenzhen (CN); Yongtian Lu, Shenzhen (CN); Weiping Li, Shenzhen (CN); Haidong Wang, Shenzhen (CN); Tong Wang, Shenzhen (CN); Zhixian Mao, Shenzhen (CN); Guangshuai Zhang, Shenzhen (CN); Chengkai Luo, Shenzhen (CN); Wanfeng Shang, Shenzhen (CN); Yajing Shen, Shenzhen (CN); Zhuohua Lin, Shenzhen (CN); Riwei Zhang, Shenzhen (CN); Jianjun Long, Shenzhen (CN); Yulong Wang, Shenzhen (CN)

(21) Appl. No.: 17/568,366

(22) Filed: Jan. 4, 2022

(30) Foreign Application Priority Data
Jan. 12, 2021 (CN) 2021100367576

(51) Int. Cl. A61B 10/00; B25J 9/16; A61G 10/02

(52) U.S. Cl. CPC A61B 10/0051; A61G 10/023; B25J 9/1697

(57) ABSTRACT

A robot and a system for a medical operation of corona viruses e.g., COVID-19 sampling are provided. The robot includes a resilient connector, connecting plates connected with the resilient connector, stretchable and retractable assemblies, a driving assembly and a flexible sampling assembly; two ends of the stretchable and retractable assembly are respectively connected with adjacent two connecting plates, the stretchable and retractable assembly includes stretchable and retractable components divided into at least two stretchable and retractable groups and arranged around the resilient connector, the driving assembly drive the stretchable and retractable component to be switchably stretched or retracted, the flexible sampling assembly is configured to collect COVID-19 specimens of respiratory tract. The robot can be bent and moved freely in special-shaped human respiratory tract, realize secretions sampling of human respiratory tract and reduce pain and difficulty of examination.

### TECHNICAL FIELD

The disclosure relates to the technical field of medical apparatus and instruments, in particular to a robot and a system for a medical operation of corona viruses (for example, COVID-19) sampling.

### DESCRIPTION OF RELATED ART

Human cavity examination is one of common means for doctors to diagnose diseases, such as the examination and the mucosal secretion collection of narrow cavities such as nasal cavity, oral cavity and air pipe. However, there are many blind areas due to the bending and narrow space of the narrow cavities such as nasal cavity, oral cavity and trachea. The manipulator of the existing cavity medical operation robot is usually linear or has few bending degrees of freedom, which not only brings inconvenience to the doctor's examination, but also adds pain to the patient.

### SUMMARY OF THE DISCLOSURE

A purpose of the disclosure is to provide a robot for a medical operation of corona viruses (for example, COVID-19) sampling, which can combine multiple direct motions and bending degrees of freedom.

Another purpose of the disclosure is to provide a system for a medical operation of corona viruses (for example, COVID-19), which can reduce the pain of patients and reduce the difficulty of doctors' sampling.

In order to achieve the above technical effects, the technical scheme of the disclosure is as follows:

The robot for the medical operation of corona viruses sampling, includes: a resilient connector; a plurality of connecting plates, the plurality of connecting plates are connected with the resilient connector and arranged at intervals along a lengthwise direction of the resilient connector; a plurality of stretchable and retractable assemblies, two ends of each of the plurality of stretchable and retractable assemblies are respectively connected with adjacent two of the plurality of connecting plates, each of the plurality of stretchable and retractable assemblies includes a plurality of stretchable and retractable components arranged around the resilient connector, the plurality of stretchable and retractable components are divided into at least two stretchable and retractable groups, and each of the at least two stretchable and retractable groups includes the two stretchable and retractable components respectively arranged on opposite two sides of the resilient connector; a driving assembly, the driving assembly is configured to drive the plurality of stretchable and retractable components of the each of the plurality of stretchable and retractable assemblies to be switchably stretched or retracted; and a flexible sampling assembly, the flexible sampling assembly is penetrated in the plurality of connecting plates and the plurality of stretchable and retractable assemblies and configured to collect secretions of human respiratory tract.

Further, the plurality of stretchable and retractable components respectively include a plurality of flexible airbags, and the driving assembly includes an inflation and deflation structure.

Further, the inflation and deflation structure includes a plurality of air pipes, each of the plurality of air pipes is arranged extending along the lengthwise direction of the resilient connector, and each of the plurality of air pipes is connected with one or more of the plurality of flexible airbags.

Further, the resilient connector includes a plurality of resilient tubes successively sleeved one after another, and one end of each of the plurality of resilient tubes is connected with a corresponding one of the plurality of connecting plates.

Further, the one end of each of the plurality of resilient tubes is provided with a connecting protrusion, and the corresponding one of the plurality of connecting plates is provided with a connecting groove matched with the connecting protrusion.

Further, the number of the at least two stretchable and retractable groups is two, the two stretchable and retractable components of one of the two stretchable and retractable groups are arranged along a x-axis direction, the two stretchable and retractable components of the other one of the two stretchable and retractable groups are arranged along a y-axis direction, and the lengthwise direction of the resilient connector coincides with a z-axis direction, and the x-axis direction, the y-axis direction and the z-axis direction are mutually perpendicular directions in a coordinate system.

Further, a cross section of each of the plurality of stretchable and retractable components is fan-shaped, and correspondingly an outer peripheral surface of each of the plurality of stretchable and retractable assemblies is a circumferential surface; and an outer peripheral surface of each of the plurality of connecting plates is a circumferential surface.

Further, the flexible sampling assembly includes a flexible sampling tube and a visual perception structure, the flexible sampling tube and the visual perception structure both are protruded from an end of the resilient connector; the flexible sampling tube is configured to collect the secretions of human respiratory tract of the one end of the resilient connector; and the visual perception structure is configured to obtain image information of human tissues to guide the driving assembly to drive the plurality of stretchable and retractable assemblies to move in the human respiratory tract.

Further, the robot for the medical operation of corona viruses sampling further includes a flexible protective film, the flexible protective film is wrapped on outer peripheral surfaces of the plurality of connecting plates and the plurality of stretchable and retractable assemblies.

Further, the robot for the medical operation of corona viruses sampling is a robot for a medical operation of COVID-19 sampling.

A system for the medical operation of corona viruses sampling, includes: the robot for the medical operation of corona viruses sampling described above; the robot for the medical operation of corona viruses sampling is disposed in a negative pressure operating room; an intelligent terminal, the intelligent terminal is disposed outside the negative pressure operation room and communicated with the robot for the medical operation of corona viruses sampling; the intelligent terminal is configured to control the driving assembly to drive the plurality of stretchable and retractable components to be stretched or retracted and receive information collected by the robot for the medical operation of corona viruses sampling, and the intelligent terminal is further configured to acquire real-time image information of the negative pressure operating room.

One beneficial effect of the disclosure is: according to the robot for the medical operation of corona viruses sampling, the robot can be bent and moved freely in the special-shaped respiratory tract and realize the sampling of the secretions of human respiratory tract, thereby reducing the pain of patients and the examination difficulty of doctors.

Another beneficial effect of the disclosure is: because of the robot for the medical operation of corona viruses sampling described above, intelligent control of the robot for the medical operation of corona viruses sampling can be realized through the intelligent terminal, so that the plurality of stretchable and retractable assemblies of the robot for the medical operation of corona viruses sampling can better adapt to the narrow and special-shaped tract of human body, and be bent, stretched or retracted freely in the cavity tract, so as to reduce the pain of patients and reduce the difficulty of doctors' examination operation.

Additional aspects and advantages of the disclosure will be given in part in the following description, and some will become apparent from the following description, or known through the practice of the disclosure.

### BRIEF DESCRIPTION OF THE DRAWINGS

FIG. 1 is a schematic structural diagram of a robot for a medical operation of corona viruses sampling according to an embodiment of the disclosure.

FIG. 2 is a schematic exploded diagram of a partial structure of the robot for the medical operation of corona viruses sampling according to the embodiment of the disclosure.

FIG. 3 is a schematic exploded diagram of structures of connecting plates and stretchable and retractable assemblies according to the embodiment of the disclosure.

FIG. 4 is a schematic diagram of a partial structure the resilient connector according to the embodiment of the disclosure.

FIG. 5 is a schematic diagram of using of a system for a medical operation of corona viruses sampling according to an embodiment of the disclosure.

### REFERENCE MARK

1, resilient connector; 11, resilient tube; 12, connecting protrusion;
2, connecting plate; 21, connecting groove; 22, first connecting through hole; 23, second connecting through hole;
3, stretchable and retractable assembly; 31, stretchable and retractable component; 4, air pipe;
5, flexible sampling assembly; 51, flexible sampling tube; 52, visual perception structure; 6, outer housing;
100, robot for the medical operation of corona viruses sampling; 110, display mechanism; 120, mobile mechanism; 130, lifting mechanism.

### DETAILED DESCRIPTION OF EMBODIMENTS

In order to make the technical problems solved, the technical scheme adopted and the technical effect achieved by the disclosure clearer, the technical scheme of the disclosure is further described below in combination with the accompanying drawings and through specific embodiments.

In the description of the disclosure, unless otherwise clearly specified and limited, the terms “connected” and “fixed” shall be understood in a broad sense. For example, it can be fixed connection, removable connection, or integrated; it can be mechanical connection or electrical connection; it can be directly connected or indirectly connected through an intermediate medium; it can be the connection within two elements or the interaction relationship between two elements. For those skilled in the art, the specific meaning of the above terms in the disclosure can be understood in specific circumstances.

In the disclosure, unless otherwise expressly specified and limited, the first feature “above” or “below” of the second feature may include direct contact between the first and second features, or the first and second features may not be in direct contact, but through another feature contact between them. Moreover, the first feature is “above” of the second feature, including that the first feature is directly above and obliquely above the second feature, or only indicates that the horizontal height of the first feature is higher than that of the second feature. The first feature is “below” of the second feature, including that the first feature is directly below and obliquely below the second feature, or only indicates that the horizontal height of the first feature is less than that of the second feature.

It should be understood that the terms “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, “circumferential” to indicate the azimuth or positional relationship based on the azimuth or positional relationship shown in the attached drawings, which is only for the convenience of describing the disclosure and simplifying the description, rather than indicating or implying that the device or element must have a specific azimuth, be constructed and operated in a specific azimuth, so it cannot be understood as a limitation of the disclosure. In the description of the disclosure, unless otherwise specified, “multiple” or “the plurality of” means two or more. In addition, the terms “first” and “second” are only used to distinguish in description and have no special meaning.

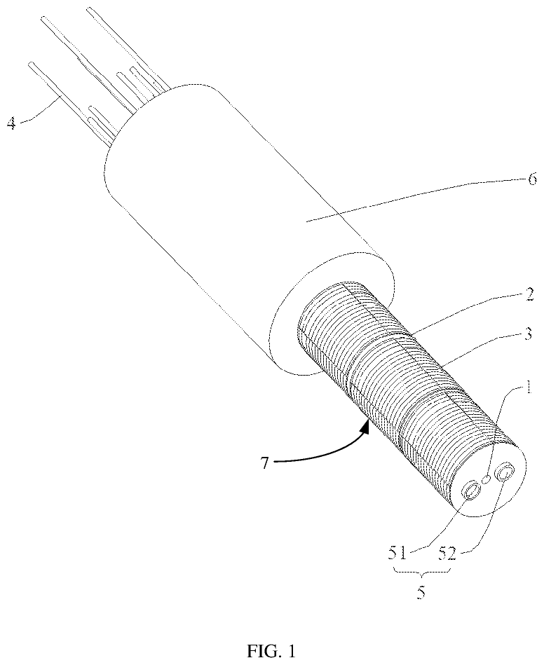

Next, referring to FIG. 1 to FIG. 5, a robot for the medical operation of corona viruses sampling of an embodiment of the disclosure is a robot for a medical operation of COVID-19 sampling as an example, a specific structure of a robot for a medical operation of corona viruses sampling is described.

As shown FIG. 1 to FIG. 5, FIG. 1 show the robot for the medical operation of corona viruses sampling, the robot for the medical operation of corona viruses sampling includes: a resilient connector 1, a plurality of connecting plates 2, a plurality of stretchable and retractable assemblies 3, a driving assembly and a flexible sampling assembly 5. The plurality of connecting plates 2 are connected with the resilient connector 1 and arranged at intervals along a lengthwise direction of the resilient connector 1. two ends of each stretchable and retractable assembly 3 are respectively connected with adjacent two connecting plates 2, each stretchable and retractable assembly 3 includes a plurality of stretchable and retractable components 31 arranged around the resilient connector 1, the plurality of stretchable and retractable components 31 are divided into at least two stretchable and retractable groups, and each of the at least two stretchable and retractable component groups includes the two stretchable and retractable components 31 respectively arranged on opposite two sides of the resilient connector 1. The driving assembly is configured to drive the stretchable and retractable component 31 to be switchably stretched or retracted. The flexible sampling assembly 5 is penetrated in the plurality of connecting plates 2 and the plurality of stretchable and retractable assemblies 3 and configured to collect secretions of human respiratory tract.

It can be understood that when the drive assembly drives one stretchable and retractable component 31 to be stretched, the one stretchable and retractable component 31 will drive the resilient connector 1 to be stretched and drive one connecting plate 2 connected with the one stretchable and retractable component 31 to be moved in the lengthwise direction of the resilient connector 1, at the same time, the other stretchable and retractable components 31 adjacent to the one stretchable and retractable component 31 and belonging to the same stretchable and retractable assembly 3 are still maintained in the original length, and the other stretchable and retractable components 31 are also connected with the one connecting plate 2, so that the stretchable and retractable assembly 3 where the one stretchable and retractable component 31 is located is be bent; in addition, when the driving assembly drives all the stretchable and retractable components 31 in the one stretchable and retractable assembly 3 to be stretched or retracted at the same time, the one stretchable and retractable assembly 3 can be correspondingly stretched or retracted in the lengthwise direction of the resilient connector 1, that is, the displacement of the connecting plate 2 connected with the one stretchable and retractable assembly 3 in the lengthwise direction of the resilient connector 1 is realized. Thus, by driving the different stretchable and retractable components 31 of the stretchable and retractable assembly 3 to be stretched or retracted by the driving assembly, the stretching, retracting and bending of the plurality of stretchable and retractable assemblies 3 can be realized, and the plurality of stretchable and retractable assemblies 3 make different attitude changes under the action of the driving assembly, so that the plurality of stretchable and retractable assemblies 3 can have a combination of multiple degrees of freedom of direct motion and bending, so as to effectively realize the diversified control of the shape of the robot, then, the robot can enter, exit and bend freely in the special-shaped and different-width cavity tracts in the human body, and reach the sampling place in the human body. Because of the flexible sampling assembly 5 is flexible, it can generate the same bending and displacement under the driving of the plurality of stretchable and retractable assemblies 3, and finally reach the sampling place, so that the robot can complete tissue sampling in human body cavity, such as mucous membrane and mucous membrane secretions, so as to obtain samples of the secretions of human respiratory tract, so as to facilitate corona viruses detection of samples. It can significantly reduce the pain of patients caused by sampling the secretions of human respiratory tract, and greatly reduce the difficulty of doctors' inspection operation.

According to this embodiment, the robot for medical operation of corona viruses sampling can be bent and moved freely in the special-shaped respiratory tract of the human body and realize the sampling of the internal tissues of the human body, thereby relieving the patient's examination pain and reducing the difficulty of the doctor's examination.

Exemplary, the robot for medical operation of corona viruses sampling in this embodiment is especially suitable for the examination of narrow cavity tracts such as nasal cavity, oral cavity, air pipe 4, and the collection of mucous secretions from nasal cavity, oral cavity, respiratory tract and air pipe 4, so as to facilitate the collection and testing of corona viruses samples.

In some embodiment, the stretchable and retractable component 31 includes an flexible airbag, and the driving assembly includes an inflation and deflation structure.

It can be understood that the stretching and retracting of the flexible airbag is achieved relatively simple and convenient, and the radial size of the flexible airbag changes very little in the stretching and retracting process, so that the stretchable and retractable component 31 will not interfere with the special-shaped respiratory tract during the stretching and retracting process, which clearly prevents the medical accident of the stretchable and retractable component 31 stuck in the human cavity tract during the stretching and retracting process. At the same time, the inflation and deflation structure is also conducive to extending in all stretchable and retractable assemblies 3, so as to realize the driving the stretchable and retractable component 31 extending from the human body to be stretched or retracted.

In some embodiment, as shown in FIG. 1, the inflation and deflation structure includes a plurality of air pipes 4, each of the plurality of air pipes 4 is arranged extending along the lengthwise direction of the resilient connector 1, and each air pipe 4 is connected with the one or more of the plurality of flexible airbags.

It can be understood that the plurality of air pipes 4 can respectively inflate and deflate the flexible airbags, so as to realize the separate control of the stretching and retracting of each stretchable and retractable component 31 by the driving assembly, which is conducive to improving the accuracy of the stretching and retracting of the stretchable and retractable assembly 3, so as to improve the bending accuracy of the whole robot, so as to enable the robot to move in the complex special-shaped respiratory tract of the human body, the collision phenomenon between the robot and human tissue is reduced, and the examination pain of patients is further reduced.

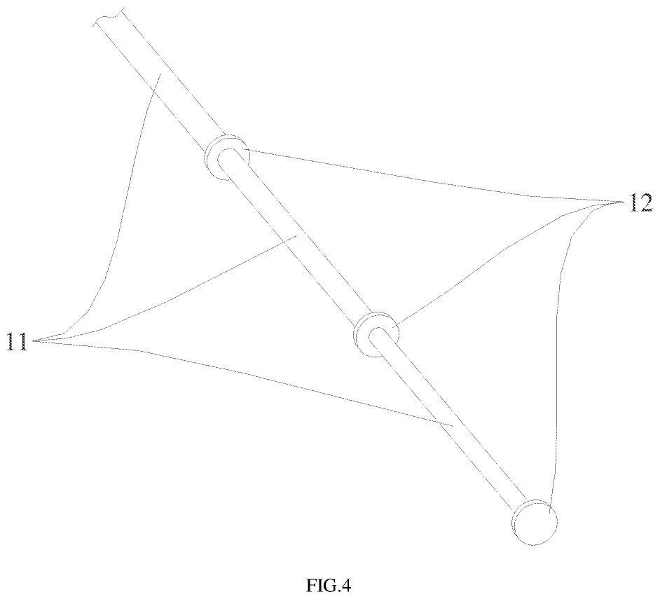

In some embodiment, as shown in FIG. 4, the resilient connector 1 includes a plurality of resilient tubes 11 successively sleeved one after another, and one end of each resilient tube 11 is connected with a corresponding one connecting plate 2.

It can be understood that since each connecting plate 2 is only connected with one resilient tube 11, when the connecting plate 2 is moved along the lengthwise direction of the resilient connector 1 under the action of the stretchable and retractable component 31, it will drive the resilient tube 11 connected to it to be axially displaced relative to other resilient tubes 11, so that the bending, stretching and retracting of the single stretchable and retractable assembly 3 are realized, it can effectively prevent the stretchable and retractable assembly 3 from interfering with other stretchable and retractable assemblies 3 during bending, stretching and retracting.

For example, when the robot is located in a relatively narrow cavity tract space, the driving component can drive the stretchable and retractable assembly 3 at the end to bend and move, and then drive the next stretchable and retractable assembly 3 to bend and move, so as to facilitate the robot to pass through the narrow cavity tract space; at the same time, when the robot is located in a relatively wide cavity tract space, the driving assembly can drive the stretchable and retractable assembly 3 closed to the driving assembly to bend, so as to drive other stretchable and retractable components to swing in the cavity tract space, which significantly improves the flexibility of the robot and is conducive to the flexible sampling assembly 5 to collect the secretions of human respiratory tract in the cavity. In addition, since each resilient tube 11 is only connected with the one connecting plate 2, during the actual operation of the robot, the resilient tube 11 can also be driven to move in the axial direction to realize the stretching and retracting of the whole stretchable and retractable assembly 3, so as to improve the operation convenience of the robot.

Of course, in the embodiment, the amount of the stretching and retracting of the plurality of resilient tubes 11 successively sleeved one after another in its axial direction is small. In other embodiments of the disclosure, the resilient connector 1 can also be formed as an elastic structure that can not only realize being bent, but also being stretched and retracted in the axial direction, which can enable the driving assembly to realize the bending, stretching and retracting of the single stretchable and retractable assembly 3. Thus, the actual structure of the resilient connector 1 can be determined according to the actual demand without specific limitation.

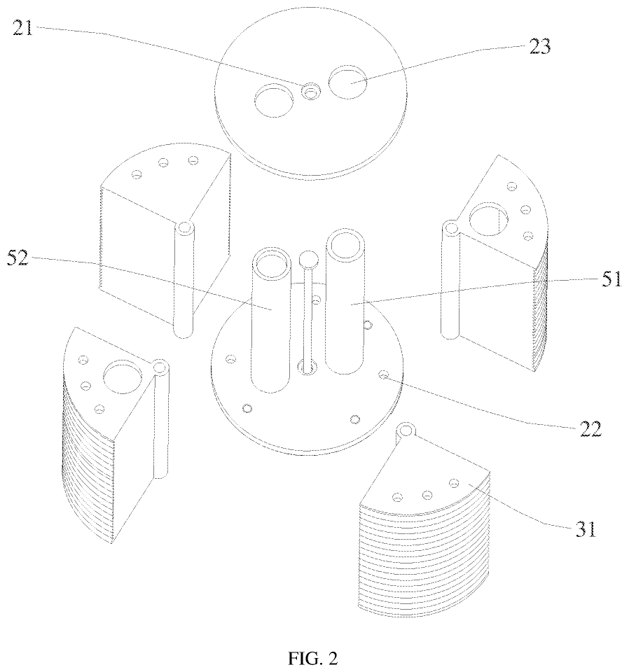

In some embodiment, as shown in FIG. 2 to FIG. 4, one end of the resilient tube 11 is provided with a connecting protrusion 12, and the corresponding connecting plate 2 is provided with a connecting groove 21 matched with the connecting protrusion 12.

It can be understood that the setting of the connecting protrusion 12 and the connecting groove 21 can better realize the stable connection between the resilient tube 11 and the connecting plate 2, so that the stretchable and retractable assembly 3 can drive the resilient tube 11 to complete the attitude change of stretching, retracting or bending at the same time through the movement of the connecting plate 2 during stretching, retracting or bending, so as to ensure the reliability of the shape change of the robot, the change flexibility of the robot is improved.

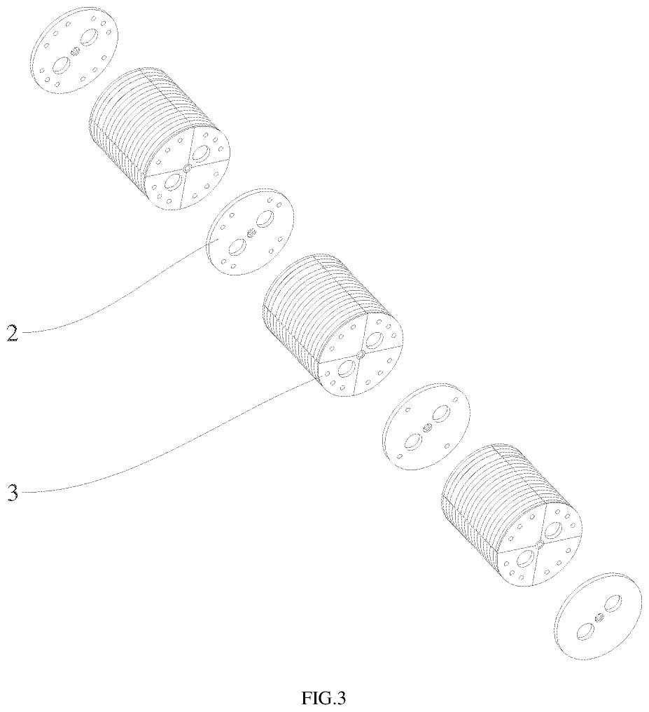

In some specific embodiments, as shown in FIG. 2, the connecting plate 2 is provided with first connecting through holes 22 and second connecting through holes 23, the first connecting through hole 22 is matched with the air pipe 4, and the second connecting through hole 23 is matched with the flexible sampling assembly 5. In addition, as shown in FIG. 3, according to the specific distribution position of each connecting plate 2 in the axial direction of the resilient connector 1, the number of the first connecting through holes 22 disposed on the connecting plate 2 is also different. Specifically, the number of the first connecting through holes 22 gradually decreases along the direction close to the flexible sampling assembly 5, so that each stretchable and retractable assembly 3 can realize stretching and retracting control through the separate driving assembly, thus, the bending and motion accuracy of the robot can be better improved.

In some embodiment, the number of the at least two stretchable and retractable groups is two, the two stretchable and retractable components 31 of one of the two stretchable and retractable groups are arranged along a x-axis direction, the two stretchable and retractable components 31 of the other one of the two stretchable and retractable groups are arranged along a y-axis direction, and the lengthwise direction of the resilient connector 1 coincides with a z-axis direction, and the x-axis direction, the y-axis direction and the z-axis direction are mutually perpendicular directions in a coordinate system.

It can be understood that when one of the two stretchable and retractable components 31 arranged along the x-axis direction is stretched and the other is retracted, the stretchable and retractable group will be bent in the direction of the retracted adjustable component 31, so as to the stretchable and retractable assembly 3 is bent along the x-axis direction. Similarly, by driving the two stretchable and retractable components 31 arranged along the y-axis direction to be stretched or retracted, the bending of the stretchable and retractable assembly 3 in the y-axis direction can also be realized. In addition, by simultaneously adjusting the stretchable and retractable components 31 of the two stretchable and retractable groups to be stretched or retracted, the stretchable and retractable assembly 3 can be bent in the included angle direction between the x-axis direction and the y-axis direction. According to different lengths of stretching and retracting, the universal bending movement of the stretchable and retractable assembly 3 can be realized. If the four stretchable and retractable components 31 are stretched or retracted by the same displacement, the displacement of the stretchable and retractable assembly 3 in the z-axis direction can be realized. Therefore, the stretchable and retractable assembly 3 includes four stretchable and retractable components 31, which can better realize the bending of the robot, so that the robot can adapt to the special-shaped respiratory tract of the human body, so as to reduce the control difficulty and production cost of the robot on the premise of ensuring the bending reliability of the robot. Of course, in other embodiments of the disclosure, in order to further improve the bending accuracy of the robot, the stretchable and retractable assembly 3 can also include multiple stretchable and retractable groups. The more the distribution directions of the stretchable and retractable groups are, the more accurate the bending direction of the robot can be adjusted. The specific number of the stretchable and retractable groups can be determined according to the actual demand without specific limitation.

In some embodiment, as shown in FIG. 1 to FIG. 4, a cross section of the stretchable and retractable component 31 is fan-shaped, and correspondingly an outer peripheral surface of the stretchable and retractable assembly 3 is a circumferential surface, and an outer peripheral surface of the connecting plate 2 is a circumferential surface.

It can be understood that the outer peripheral surfaces of the stretchable and retractable assembly 3 and the connecting plate 2 are the circumferential surface, which can reduce the interference and collision between the robot and the human cavity tract, so as to further reduce the examination pain of the patient. The cross section of the stretchable and retractable component 31 is fan-shaped, which can facilitate the connection with the resilient connector 1 and the formation of the stretchable and retractable assembly 3 with the circular peripheral surface, and also reduce the assembly difficulty of the stretchable and retractable component 31. Of course, in other embodiments of the disclosure, the outer peripheral surface of the connecting plate 2 and the stretchable and retractable assembly 3 can also be an arc structure of ellipse without specific limitation.

In some embodiment, the flexible sampling assembly 5 includes a flexible sampling tube 51 and a visual perception structure 52, the flexible sampling tube 51 and the visual perception structure 52 both are protruded from an end of the resilient connector 1. The flexible sampling tube 51 is configured to collect the secretions of human respiratory tract at the one end of the resilient connector 1. The visual perception structure 52 is configured to obtain image information of human tissues to guide the driving assembly to drive the plurality of stretchable and retractable assemblies 3 to move in the human respiratory tract.

It can be understood that the visual perception structure 52 can facilitate the collection of the image information of human tissues, so that the doctor can use the flexible sampling tube 51 to complete the sampling operation according to the image information, and also enable the doctor to obtain the health status of human tissue according to the image information. In addition, the visual perception structure 52 can also realize the visual navigation function, which is convenient for doctors to operate and control the motion of the robot, and is conducive to reducing the interference between the robot and human tissue.

For example, in the embodiment, the visual perception structure 52 includes a flexible endoscope, which is arranged close to the front end of the visual perception structure 52. Of course, in other embodiments of the disclosure, the visual perception structure 52 can also be other types, as long as the function of collecting image information of human tissues can be realized without specific limitation.

In some embodiments, as shown in FIG. 1, the robot for the medical operation of corona viruses sampling further includes an outer housing 6, and the outer housing 6 is located at one end of the plurality of stretchable and retractable assemblies 3 far away from the flexible sampling assembly 5, and the outer housing 6 can cover the structure of the driving assembly and the resilient connector 1, thereby enabling the robot to be fixed on the medical instrument to complete the sampling operation.

In some embodiment, as shown in FIG. 1, the robot for the medical operation of corona viruses sampling further includes a flexible protective film 7, the flexible protective film is wrapped on outer peripheral surfaces of the plurality of connecting plates 2 and the plurality of stretchable and retractable assemblies 3.

It can be understood that the flexible protective film has a smooth surface, which can significantly reduce the friction between the stretchable and retractable assembly 3 and the connecting plate 2 and the human body when moving in the human cavity tract, not only reduce the discomfort of patients, but also improve the displacement and bending smoothness of the plurality of stretchable and retractable assemblies 3. In addition, the flexible protective film can also prevent the internal environmental medium of the human body to enter the stretchable and retractable assembly 3, so as to prevent the medium to interfere the movement of the stretchable and retractable assembly 3 or damaging the stretchable and retractable assembly 3, so as to ensure the use safety of the robot.

In some embodiments, the robot for the medical operation of corona viruses sampling further includes a force sensing mechanism, and the force sensing mechanism is arranged on the outer peripheral surface of the plurality of connecting plates 2, so as to facilitate the collection of the interaction force between the stretchable and retractable assemblies 3 and the human body in the process of movement, thereby realizing the safe interaction function in the special-shaped respiratory tract, and preventing the robot from exerting too much force on the human body and causing discomfort and even injury to patients.

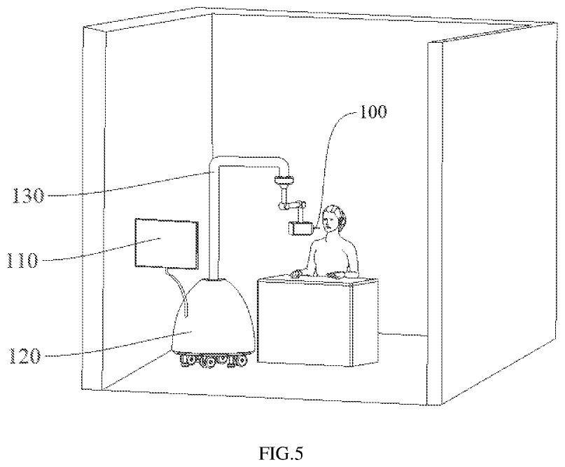

As shown in FIG. 5, an embodiment of the disclosure provides a system for the medical operation of corona viruses (for example, COVID-19) sampling, including the robot 100 for the medical operation of corona viruses (for example, COVID-19) sampling described above and an intelligent terminal. The robot 100 for the medical operation of corona viruses sampling is located in a negative pressure operating room. The intelligent terminal is located outside the negative pressure operation room and communicated with the robot 100 for the medical operation of corona viruses sampling; the intelligent terminal is configured to control the driving assembly to drive the plurality of stretchable and retractable components 31 to be stretched or retracted and receive information collected by the robot 100 for the medical operation of corona viruses sampling, and the intelligent terminal is further configured to acquire real-time image information of the negative pressure operating room.

The system for the medical operation of corona viruses sampling according to the embodiment of the disclosure can realize intelligent control of the robot 100 for medical operation of corona viruses sampling through the intelligent terminal because of the system includes the robot for the medical operation of corona viruses sampling mentioned above, so that the plurality of stretchable and retractable assemblies 3 of the robot 100 for the medical operation of corona viruses sampling can better adapt to the special-shaped and narrow tracts of the human body and can be bent, stretched or retracted freely in the cavity tract to reduce the pain of patients and the difficulty of doctors' examination. In addition, the intelligent terminal and the robot 100 for the medical operation of corona viruses sampling are respectively set outside and in of the negative pressure operating room, under the premise of ensuring the doctor's reliable operation of the robot 100 for the medical operation of corona viruses sampling and observing the patient's state, it is convenient for the patient to check the sampling operation better.

Specifically, the intelligent terminal can control the driving assembly to work according to the information fed back by the force sensing mechanism to control the stretching, retracting and bending of the plurality of stretchable and retractable assemblies 3: after the force sensing mechanism collects the interaction force between the plurality of stretchable and retractable assemblies 3, the connecting plates 2 and the human tissues, the intelligent terminal judges whether the interaction force is within the safe setting range through the preset algorithm. If the interaction force exceeds the safe setting range, the intelligent terminal automatically controls the working state of the driving assembly, and the driving stretchable and retractable assembly 3 retreats along the vertical direction of the contact force, so that the contact part of the stretchable and retractable assembly 3 and the human body tissue moves in the direction of reducing the contact force, ensuring that the robot 100 for the medical operation of corona viruses sampling is safe and interactive with the human body organization.

In some specific embodiments, as shown in FIG. 5, the narrow cavity tract medical operating system (also referred to as the system for the medical operation of corona viruses sampling) further includes a display mechanism 110, a mobile mechanism 120 and a lifting mechanism 130, the lifting mechanism 130 is arranged on the mobile mechanism 120, and the robot 100 for the medical operation of corona viruses sampling is installed on the lifting mechanism 130, and the lifting mechanism 130 can lift to adjust the height of the robot 100 for the medical operation of corona viruses sampling to adapt to the height of different patients; rollers are arranged at the bottom of the moving mechanism 120 to facilitate movement; the display mechanism 110 is configured to display the human tissue image obtained by the visual perception structure 52, so that both doctors and patients can observe the examination process at the same time, so as to facilitate effective communication between the two sides.

A system for the medical operation of corona viruses sampling of a specific embodiment of the disclosure is described below in reference to FIG. 1 to FIG. 5.

The system for the medical operation of corona viruses sampling used in this embodiment includes the robot for the medical operation of corona viruses sampling and the intelligent terminal. The robot for the medical operation of corona viruses sampling is located in the negative pressure surgery room. The intelligent terminal is located outside the negative pressure operation room, and the intelligent terminal is connected with the robot for the medical operation of corona viruses sampling. The intelligent terminal is configured to control the driving assembly to drive the plurality of stretchable and retractable components 31 to be stretched or retracted and receive the information collected by the robot for the medical operation of corona viruses sampling, and the intelligent terminal is further configured to obtain real-time image information of the negative pressure operating room.

The robot for the medical operation of corona viruses sampling includes the resilient connector 1, the plurality of connecting plates 2, the plurality of stretchable and retractable assemblies 3, the driving assembly, the flexible sampling assembly 5 and the outer housing 6.

The resilient connector 1 includes the plurality of resilient tubes 11 successively sleeved one after another, and one end of each resilient tube 11 is connected with the corresponding connecting plate 2. The one end of the resilient tube 11 is provided with the connecting protrusion 12.

The plurality of connecting plates 2 are connected with the resilient connector 1 and arranged along the lengthwise direction of the resilient connector 1. The connecting plate 2 is provided with the connecting groove 21 matched with the corresponding connecting protrusion 12. The outer peripheral surface of the connecting plate 2 is the circumferential surface.

The two ends of each stretchable and retractable assembly 3 are respectively connected with two adjacent connecting plates 2. The stretchable and retractable assembly 3 includes the plurality of stretchable and retractable components 31 arranged around the resilient connector 1. The plurality of stretchable and retractable components 31 are divided into two groups. The two stretchable and retractable components 31 of one stretchable and retractable group are arranged along the x-axis direction and the two stretchable and retractable components 31 of the other stretchable and retractable group are arranged along the y-axis direction, The lengthwise direction of the resilient connector 1 coincides with a z-axis direction, and the x-axis direction, the y-axis direction and the z-axis direction are mutually perpendicular directions in a coordinate system. The cross section of the stretchable and retractable component 31 is fan-shaped and correspondingly the outer peripheral surface of the stretchable and retractable assembly 3 is a circumferential surface.

The driving assembly can drive the stretchable and retractable component 31 to be stretched or retracted. The driving assembly includes the inflation and deflation structure, the inflation and deflation structure includes the plurality of air pipes 4, the air pipe 4 is arranged extending along the lengthwise direction of the resilient connector 1, and each air pipe 4 is connected with one or more of the plurality of flexible airbags.

The flexible sampling assembly 5 is penetrated in the plurality of connecting plates 2 and the plurality of stretchable and retractable assemblies 3, and the flexible sampling assembly 5 is configured to collect the secretions of human respiratory tract. The flexible sampling assembly 5 includes the flexible sampling tube 51 and the visual perception structure 52. Both the flexible sampling tube 51 and the visual perception structure 52 are protruded from the end of the resilient connector 1. The flexible sampling tube 51 is configured to collect the secretions of human respiratory tract of the end of the resilient connector 1. The visual perception structure 52 is configured to obtain image information of human tissues to guide the driving assembly to drive the plurality of stretchable and retractable assemblies 3 to move in the human respiratory tract.

The outer housing 6 is arranged at one end of the plurality of stretchable and retractable assemblies 3 far away from the flexible sampling assembly 5, and the outer housing 6 covers the structure of the driving assembly, the resilient connector 1, etc.

In the description of this specification, the description with reference to the terms “some embodiments”, “other embodiments”, etc. means that the specific features, structures, materials or features described in connection with the embodiment or example are included in at least one embodiment or example of the disclosure. In this specification, the schematic expression of the above terms does not necessarily refer to the same embodiment or example. Further, the specific features, structures, materials or features described may be combined in a suitable manner in any one or more embodiments or examples.

The above content is only a preferred embodiment of the disclosure. For ordinary technicians in the art, there will be changes in the specific implementation mode and application scope according to the idea of the disclosure. The content of the specification should not be understood as a limitation of the disclosure.

What is claimed is:

1. A robot for a medical operation of corona viruses sampling, comprising:
a resilient connector (1);
a plurality of connecting plates (2), wherein the plurality of connecting plates (2) are connected with the resilient connector (1) and arranged at intervals along a lengthwise direction of the resilient connector (1);
a plurality of stretchable and retractable assemblies (3), wherein two ends of each of the plurality of stretchable and retractable assemblies (3) are respectively connected with adjacent two of the plurality of connecting plates (2), each of the plurality of stretchable and retractable assemblies (3) comprises a plurality of stretchable and retractable components (31) arranged around the resilient connector (1), the plurality of stretchable and retractable components (31) are divided into at least two stretchable and retractable component groups, and each of the at least two stretchable and retractable component groups comprises the two stretchable and retractable components (31) respectively arranged on opposite two sides of the resilient connector (1);
a driving assembly, configured to drive the plurality of stretchable and retractable components (31) of the each of the plurality of stretchable and retractable assemblies (3) to be switchably stretched or retracted; and
a flexible sampling assembly (5), penetrated in the plurality of connecting plates (2) and the plurality of stretchable and retractable assemblies (3) and configured to collect secretions of human respiratory tract.

2. The robot for the medical operation of corona viruses sampling according to claim 1, wherein the plurality of stretchable and retractable components (31) respectively comprise a plurality of flexible airbags, and the driving assembly comprises an inflation and deflation structure.

3. The robot for the medical operation of corona viruses sampling according to claim 2, wherein the inflation and deflation structure comprises a plurality of air pipes (4), each of the plurality of air pipes (4) is arranged extending along the lengthwise direction of the resilient connector (1), and each of the plurality of air pipes (4) is connected with one or more of the plurality of flexible airbags.

4. The robot for the medical operation of corona viruses sampling according to claim 1, wherein the resilient connector (1) comprises a plurality of resilient tubes (11) successively sleeved one after another, and one end of each of the plurality of resilient tubes (11) is connected with a corresponding one of the plurality of connecting plates (2).

5. The robot for the medical operation of corona viruses sampling according to claim 4, wherein the one end of each of the plurality of resilient tubes (11) is provided with a connecting protrusion (12), and the corresponding one of the plurality of connecting plates (2) is provided with a connecting groove (21) matched with the connecting protrusion (12).

6. The robot for the medical operation of corona viruses sampling according to claim 1, wherein the number of the at least two stretchable and retractable groups is two, the two stretchable and retractable components (31) of one of the two stretchable and retractable groups are arranged along a x-axis direction, the two stretchable and retractable components (31) of the other one of the two stretchable and retractable groups are arranged along a y-axis direction, the lengthwise direction of the resilient connector (1) coincides with a z-axis direction, and the x-axis direction, the y-axis direction and the z-axis direction are mutually perpendicular directions in a coordinate system.

7. The robot for the medical operation of corona viruses sampling according to claim 1, wherein a cross section of each of the plurality of stretchable and retractable components (31) is fan-shaped, and correspondingly an outer peripheral surface of each of the plurality of stretchable and retractable assemblies (3) is a circumferential surface; and an outer peripheral surface of each of the plurality of connecting plates (2) is a circumferential surface.

8. The robot for the medical operation of corona viruses sampling according to claim 1, wherein the flexible sampling assembly comprises a flexible sampling tube (51) and a visual perception structure (52), the flexible sampling tube (51) and the visual perception structure (52) both are protruded from an end of the resilient connector (1);
wherein the flexible sampling tube (51) is configured to collect the secretions of human respiratory tract of the one end of the resilient connector (1); and
wherein the visual perception structure (52) is configured to obtain image information of human tissues to guide the driving assembly to drive the plurality of stretchable and retractable assemblies (3) to move in the human respiratory tract.

9. The robot for the medical operation of corona viruses sampling according to claim 1, wherein the robot for the medical operation of corona viruses sampling further comprises a flexible protective film, wrapped on outer peripheral surfaces of the plurality of connecting plates (2) and the plurality of stretchable and retractable assemblies (3).

10. The robot for the medical operation of corona viruses sampling according to claim 1, wherein the robot for the medical operation of corona viruses sampling is a robot for a medical operation of COVID-19 sampling.

11. A system for the medical operation of corona viruses sampling, comprising:
the robot for the medical operation of corona viruses sampling according to claim 1, wherein the robot for the medical operation of corona viruses sampling is disposed inside a negative pressure operating room; and
an intelligent terminal, disposed outside the negative pressure operation room and communicated with the robot for the medical operation of corona viruses sampling, wherein the intelligent terminal is configured to control the driving assembly to drive the plurality of stretchable and retractable components (31) to be stretched or retracted and receive information collected by the robot for the medical operation of corona viruses sampling, and the intelligent terminal is further configured to acquire real-time image information of the negative pressure operating room.
